(19) United States
(12) Patent Application Publication
(10) Pub. No.: US 2026/0325474 A1
(43) Pub. Date: Oct. 22, 2026

(54) Nutraceutical Method for Hypothalamic Adiposity Switching Through Targeted GI-Lipid Stimulation

(71) Applicants: Terry Earl Brady, The Valley (AI); Anthony Lee Dellinger, Burlington, NC (US); Lowell Hughes, The Valley (AI); Melinda K. M. Goddard, The Valley (AI); Abed Alqader Ibrahim, Greensboro, NC (US)

(72) Inventors: Terry Earl Brady, The Valley (AI); Anthony Lee Dellinger, Burlington, NC (US); Lowell Hughes, The Valley (AI); Melinda K. M. Goddard, The Valley (AI); Abed Alqader Ibrahim, Greensboro, NC (US)

(21) Appl. No.: 19/180,505

(22) Filed: Apr. 16, 2025

(51) Int. Cl. A61K 31/201; A61K 9/48; A61K 9/50; A61K 31/202; A61K 31/23; A61K 31/661; A61K 31/722; A61K 36/63; A61K 36/68

(52) U.S. Cl. CPC A61K 31/201; A61K 9/4816; A61K 9/5073; A61K 31/202; A61K 31/23; A61K 31/661; A61K 31/722; A61K 36/63; A61K 36/68

(57) ABSTRACT

Throughout human evolution, survival was tied to the neurobiology of cellular energy requirements, with circadian caloric consumption that signaled satiety or hunger to the hypothalamus. Scarcity conditions reinforced episodic adiposity accumulation primarily to survive famine until approximately 12,000 years ago. However, early relative abundance was nonetheless insufficient to trigger widespread obesity, as cultures centered around a single full meal per day. While evolving primarily to address scarcity, gastrointestinal lipid signaling can stimulate the hypothalamus to accumulate or oxidize adipose tissues. In turn, the hypothalamus can be stimulated to enter one state or the other through sustained lipid dosage or absorption in the small intestine as a metabolic switch. This invention involves lipid matter or isolates that induce neurobiological switching, presuming a caloric bias of abundance or scarcity, for the purpose of weight management. This switching mechanism is most acute from birth to infancy but less pronounced in adulthood.

### FIELD OF INVENTION

This proposed invention relates to human metabolic regulation, specifically how the gastrointestinal (GI) tract serves as a pathophysiological pathway to signal the hypothalamus to initiate adipose tissue accumulation or oxidation. The invention applies sustained lipid dosage or absorption in the small intestine to signal the hypothalamus to enter one state or the other, as a metabolic switch. The utility addresses long-standing challenges in weight control by targeting the underlying mechanism for caloric retention or consumption.

### BACKGROUND

Overweight and obesity in most nations has increasingly challenged healthcare systems and societal norms since the mid-1900s to have become a global epidemic today. However, sustained, excessive weight remained historically uncommon until its emergence in Greek and Roman societies, and then only among a small elite. As such, evolutionary evidence suggests that humans developed a robust survival mechanism, governed by the hypothalamus, to maintain homeostasis, resisting both excessive adipose accumulation and extreme fat burning. This mechanism functions prominently in infancy and childhood before moderating thereafter, as the hypothalamus responds to changes in circulating fatty acids and regulates appetite and related metabolic responses throughout adulthood (Lam, T. K. T., et. al., “Hypothalamic Sensing of Fatty Acids,” Nature Neuroscience, May 2005). Moreover, hypothalamic switching can span weeks for equilibration, with resistance plateaus that are disproportionate to caloric mathematics, reflecting this GI switch pathophysiology.

Notably, the absence of gastrointestinal lipid absorption profoundly disrupts metabolism, as a result of an essential animal fat signaling pathway connecting the gut to the hypothalamus. Physiologically, this response is thought to originate from early diets reliant on animal fat, which provided dense caloric value in short daylight hunting and gathering windows. Compared to the bulk of plant matter required to yield equivalent energy, a single grub offered advantages in fatty acid delivery. And while mammoth hunts may have occasionally yielded more than early humans could consume before spoilage, their circadian pattern of one primary meal near nightfall, followed by cessation of both eating and further activity at dusk, reduced opportunities for fat accumulation.

Seasonal variety, diverse food sources, and an evolutionary elongation of the GI tract favored such intermittent consumption, a pattern still evident in aboriginal societies today. Paleoanthropological studies point to three significant epochs of bipedal evolution: A 30-million-year lineage of primates, the hominin stage, including Homo erectus, and finally the rise of Homo sapiens (300,000 to 500,000 years ago). One theory credits bipedalism with conferring dominance on humans over their surroundings, supported by an omnivorous diet. The use of fire, approximately 20,000 years ago, also made animal fats more digestible, fueling neurobiological development and the advancement of communal structures. In turn, over millennia, this synergy of nutrition, neurobiology, and communal behavior contributed to the development of both spoken and written languages and complex societies. Nonetheless, dietary abundance remained sporadic. Only after development of Mesopotamian agriculture (about 12,000 years ago) did a more reliable surplus of processed grains arise, yet even then, prolonged labor, scarce leisure, and the tradition of sleeping through dark hours maintained relatively stable body composition for most peoples.

The overconsumption that ultimately emerged in the Greek and Roman upper classes (e.g., exotic feasting and purging customs) nonetheless exposed the capacity of the human gastrointestinal tract to handle excessive intake without immediate breakdown of metabolic regulation. Roman physicians noted that chronic overeating eventually led to health problems, however, they attributed them to a lack of restraint rather than systematic metabolic failures.

While these dynamics resulted in sporadic obesity in Greco-Roman cultures among elites with the means to overindulge, “robust” body types were occasionally culturally favored thereafter, such as for men as soldiers or women planning children, but the current obesity epidemic is unique in human history. As such, while the English gentry adopted customs for eating hearty breakfasts in the last two hundred years, most cultures continued to eat lightly or skipped early-morning meals entirely, then ceased dining before nightfall. This pattern matched the ancestral physiology that seldom encountered persistent excess.

In fact, until the mid-twentieth century, food scarcity, circadian rhythms, and extended digestion intervals synergized for most individuals in all populations to maintain generally healthy weight for both genders and all age groups. Yet, in the modern setting, with continuous access to calorie-dense, often processed foods, these inherent mechanisms have led to metabolic dysregulation and prevalent obesity. Even a modest annual weight gain (e.g., 0.5%) accumulates over decades.

Now in the 21^{st} century, nearly 75% of most global populations have become overweight, prompting the American Medical Association in 2012 to classify obesity as a disease, albeit one lacking clear etiology or treatment solutions. As such, these historically recent cultural, economic and food processing shifts driving near-constant abundance have outpaced genetic adaptation, leading to the evolving obesity epidemic and a paucity of effective weight management solutions.

Moreover, erratic caloric intake has also been observed to stimulate abnormal appetite perceptions of “starvation” in lieu of the hypothalamus metabolizing adipose tissues in homeostasis. That is, once caloric intake is withheld for an extended period, muscle and other vital tissues may be oxidized along with adipose deposits. Human obesity is thus linked to a breakdown in the natural GI-hypothalamus signaling that evolved to regulate fat storage under conditions of scarcity.

The hypothalamus response to the presence or absence of dietary fat has also been documented in the phenomenon known as “rabbit starvation.” Past accounts, from ancient Rome to modern wilderness survival reports, have detailed severe metabolic dysfunction and even death following prolonged consumption of lean protein without sufficient fat intake. Symptoms include extreme cravings for fats and signs of protein poisoning (i.e., severe ketosis and metabolite overload), presumably due to endocrine and neurobiological imbalances governed by the gut-brain-hypothalamus axis.

In short, obesity has arisen from disrupted signaling between the presence or absence of GI lipids and the hypothalamus rather than from genetic predisposition. Genetics cannot account for the global rise in overweight populations that has occurred in decades, not millennia, as required for an evolutionary shift.

In turn, efforts to reduce bodily weight have consistently failed due to inherent resistance to changes in metabolic pathways. Conventional dieting, which typically involves caloric restriction through portion limitations and/or less calorie-dense food group substitutions, rarely enables a sustainable metabolic shift for adiposity oxidation. Attempts to manipulate perceived hunger have also demonstrated suboptimal outcomes using synthetic GLP-1 analogs (GLP-1 receptor agonists, GLP-1RA) that extend its half-life to several days, wherein most patients lose some weight, while others abandon the therapy due to side effects. With regard to this mechanism, natural GLP-1 has a short half-life (~2 minutes) and thus, transiently helps regulate hunger; however, the synthetic analogs may also disrupt GI-based switching between fat storage and oxidation. Such robust hormones can also diffuse into non-target pathways, diminishing the capacity of the hypothalamus to perform its toggling function.

Despite extensive clinical use of GLP-1RAs for appetite regulation and weight reduction, the weight loss from using these agents nonetheless tends to plateau. Clinical data indicate that many morbidly obese individuals fail to achieve significant or sustained weight loss from GLP-1RA therapy alone. This is partially due to a physiological threshold, below which further appetite suppression does not translate into continued fat oxidation, and a metabolic ceiling, beyond which additional dosing does not yield proportional reductions in adiposity. These limitations suggest the need for innovation that can engage non-appetite-based mechanisms, such as GI signaling to the hypothalamus, to directly modulate adiposity switching.

U.S. Pat. No. 4,598,089 (Leucine derivatives) by Hoffmann-La Roche Inc. discloses XENICAL® (Orlistat, tetrahydrolipstatin), a pancreatic lipase inhibitor that reduces dietary fat absorption for weight management. Orlistat primarily addresses the enzymatic breakdown of fats, whereas the claimed method harnesses an evolved gut-hypothalamus axis to sustain metabolic changes through signal-based pathways. Unlike the malabsorption approach of Orlistat, the present invention leverages targeted GI-lipid presence or absence to manipulate a neurobiological “switch” to toggle between adiposity storage and oxidation. Thus, the present invention presents a more natural and potentially lower-impact alternative for weight modulation than that of lipase activity inhibition as taught by the Orlistat patent.

Plant-based cholesterol-lowering compositions, as taught in U.S. Pat. No. 6,933,291, block or reduce intestinal cholesterol absorption (e.g., phytosterols or phytostanols) and/or inhibit endogenous cholesterol biosynthesis. The present invention diverges from the prior art by focusing on hypothalamic adiposity switching via GI-lipid signaling and does not rely solely on interference with cholesterol uptake or production. This proposed invention would stimulate fat accumulation or oxidation by altering lipid availability in the small intestine and engaging a neurobiological mechanism that is distinct from conventional plant-based methods for reducing serum cholesterol.

Given the genetic distribution of somatotypes, a pathophysiological pathway that regulates the intake of dietary energy, either storing it as adipose tissue or utilizing it for essential physiological functions, can be observed in most human populations. That is, a small share of the population continually may struggle to achieve a healthy weight, whereas many others constantly find challenges to avert an inexorable path toward obesity. As the range of somatotypes is determined by the responsiveness of the hypothalamus to the dynamics of nutrition, biology, and anatomy, the proposed invention leverages the neurobiology of the hypothalamus to initiate adipose storage or oxidation in response to abundance or scarcity of lipids in the small intestine for weight management.

### SUMMARY OF THE INVENTION

Against this backdrop, the capacity to switch from fat storage to fat burning can thus be manipulated by prolonged introduction or extraction of lipids in the small intestine. The primary embodiment of the present invention discloses a nutraceutical to modulate adiposity switching through direct gastrointestinal stimulation. This is achieved by administering of one of two embodiments delivering a specific active and secondary ingredients in an engineered, enteric-coated oral form to overcome hypothalamic resistance as a method of long-term weight management. That is, to “break a plateau” for weight loss or accumulation.

Thus, in alternative embodiments, the formulation may be tailored to support particular metabolic objectives, including weight gain in individuals with wasting syndrome or weight loss in those with metabolic resistance. Alternative formulations and dosage could thus restore hypothalamic homeostasis by modulating neuroendocrine pathways impacted by caloric excess, deficiency, pharmaceutical agents, and/or environmental disruption.

In the context of this invention, fat burning, or oxidation, refers to the process by which stored adipose tissue is converted into energy and metabolized. This process involves the breakdown of fatty acids through beta-oxidation in mitochondria, producing acetyl-CoA, which then enters the Krebs cycle to generate adenosine triphosphate (ATP), the primary energy currency of all living cells. ATP fuels essential physiological functions, including locomotion, cognition, thermoregulation, and homeostasis. The mechanism of metabolic switching facilitated by this invention thus enables the body to toggle between fat accumulation, typically induced during periods of caloric abundance, and fat oxidation, triggered during perceived scarcity or through GI lipid depletion.

Promoting fat oxidation means shifting the body from a state of passive energy storage to an active utilization of stored fat, resulting in weight reduction driven by endogenous energy metabolism rather than caloric restriction alone. This mechanistic distinction is particularly relevant when compared to GLP-1RAs, which primarily act through appetite suppression and do not consistently promote actual fat oxidation, especially in individuals with high metabolic resistance or morbid obesity.

A corresponding clinical embodiment of the fat accumulation formulation may be optimized to reverse or stabilize wasting disorders such as cachexia, malnutrition, and unintentional weight loss associated with chronic illness. By delivering a consistent lipid signal to the small intestine, the lipid-dense formulation can stimulate adiposity accumulation in individuals experiencing pathological fat depletion. In parallel, the neurobiological regulation offered through hypothalamic switching may help recalibrate dysfunctional appetite and satiety signaling, thus providing a novel mechanism to ameliorate disordered eating behaviors, potentially including cycles of bingeing or chronic restriction.

The invention thus comprises a pair of nutraceutical formulations for modulating adiposity switching via direct gastrointestinal stimulation. The distinct formulations are engineered to promote the transition between adiposity accumulation or fat oxidation by delivering a defined blend of natural ingredients in an enteric-coated capsule or tablet.

In the fat-oxidation formulation, targeted release to the small intestine is achieved using an alginic acid coating for protection from the acidic gastric environment and ensures optimal release for lipid signaling. The alginic acid protective coating hydrates the duodenum, traps bile salts, restricts lipid emulsification and promotes fat-binding. However, the adiposity-accumulation formulation excludes alginate, as the bile-sequestering effect would hinder performance. In this embodiment, a three-layer, pH-triggered system sequentially releases active ingredients in the duodenum, mid-jejunum and distal ileum. This pH-responsive coating in combination with active ingredient layering would preserve bile activity and maximize lipid absorption to drive anabolic hypothalamic signaling. In other embodiments, an enteric or alternative advanced encapsulation method for targeted small-intestine release may be desired and would be considered obvious to someone skilled in the art.

### DETAILED DESCRIPTION OF THE INVENTION

The present invention thus provides a nutraceutical formulation for inducing adiposity switching via direct gastrointestinal stimulation. In the weight loss embodiment of the present invention, by combining a synergistic blend of conjugated linoleic acid (CLA), chitosan-alpha-lipoic acid (ALA) complexes, psyllium husk, and optional anti-absorption agents within an enteric-coated, alginic acid capsule or tablet, the formulation achieves targeted delivery in the small intestine, enhanced bioavailability, and a dual mechanism that signals the hypothalamus to promote fat oxidation while reducing dietary fat absorption.

In the fat accumulation embodiment, the enteric-coated composition is delivered in three phases: 1) a “priming phase” containing octanoic (C8)- and decanoic (C10)-rich MCTs; 2) a “storage phase” comprising structured triglycerides enriched in sn-2 palmitate, olive oil fortified with palmitoleic acid and essential phospholipids; and 3) an “anti-catabolic phase” providing high-purity EPA/DHA. These lipids signal the hypothalamus to maximize intestinal fat absorption (associated with weight gain in growth-restricted infants and cachectic adults), enhance nitrogen balance, and act as an insulin-sensitizing lipokine that directs newly absorbed energy into adipose tissue, while limiting ectopic lipid deposition, to support controlled weight gain. Co-delivery of EPA/DHA moderate inflammatory signaling and catabolism to enable safe, net accrual of both fat and lean body mass by stabilizing hepatic lipid metabolism and averting electrolyte shifts to prevent re-feeding-syndrome-like derangements.

A sustained hypothalamic switch would be achieved through a dosage regimen spanning several days or weeks for the GI tract to consistently send lipid cues to the hypothalamus. The specific delivery system of each formulation in this invention plays a crucial role, protecting and releasing active components in the small intestine, where hypothalamus signaling occurs.

In the preferred embodiment of the present invention for fat oxidation, the supplement comprises:

| | | | |
|---|---|---|---|
| Conjugated Linoleic Acid (CLA) | 0.5-3.0 | g/day | Increase basal metabolic rates, modulate enzymatic |
| | | | processes associated with fat breakdown, and shift the |
| | | | metabolic set-point in favor of fat oxidation. |
| Chitosan | 0.25-1.0 | g/day | Chitosan is complexed with ALA to enhance its fat- |
| | | | binding capacity and improve its bioavailability. |
| Alpha-Lipoic Acid (ALA) | 0.30-0.60 | g/day | ALA is complexed with chitosan to potentiate its insulin- |
| | | | sensitizing effects by stimulating the translocation of the |
| | | | GLUT-4 transporter. |
| Psyllium Husk | 5.0-10.0 | g/day | Provides dietary fiber that enhances satiety, supports |

regular bowel movements, and modulates
gastrointestinal content viscosity to optimize nutrient
release kinetics.

This embodiment offers a dosing spectrum that may be adjusted based on individual tolerance, metabolic response, and clinical efficacy. The formulation is intended to support fat metabolism and promote favorable changes in body composition through a synergistic, multi-mechanistic mode of action that is not taught by the prior art.

The mechanism of action in the fat burning embodiment comprises four critical elements: 1) Fat Binding: Chitosan binds to dietary fats within the gastrointestinal tract, reducing fat absorption by limiting emulsification and promoting the fecal excretion of lipids. 2) Insulin Sensitization: ALA enhances insulin sensitivity by stimulating the translocation of GLUT-4 transporters to the cell membrane, thereby increasing cellular glucose uptake and promoting efficient energy utilization. 3) Fiber-Mediated Modulation: Psyllium husk prolongs satiety, aids in bowel regulation, and modulates the viscosity of gastrointestinal contents, thereby optimizing the kinetics of nutrient release. Additionally, the fiber component modulates gastrointestinal transit time, lengthens exposure of nutrients to enteroendocrine cells, which may further amplify gut-brain signaling to the hypothalamus through hormone-mediated pathways. 4) Basal Metabolic Enhancement: CLA acts as the primary active ingredient to elevate basal metabolic rates and modulate enzymatic pathways responsible for fat breakdown and storage, thereby facilitating a shift from fat accumulation to oxidation.

The primary dosage range (0.5-3.0 g/day) has been selected to span physiologically relevant thresholds for inducing hypothalamic signaling responses in both underweight and overweight individuals, based on known metabolic activity of CLA in adipose regulation. In certain embodiments, the active components may be microencapsulated to enable phased or sequential release, further mimicking lipid-signaling rhythms observed in intermittent feeding states.

A key aspect of the present invention is the specific complexation of chitosan with ALA. This is accomplished by activating the carboxyl groups of ALA, preferably via carbodiimide-mediated coupling, to form intermediates that react with the primary amine groups on chitosan. The resulting conjugate, which binds via amide linkages, protects ALA from premature oxidation and enzymatic degradation and improves its solubility and intestinal absorption due to the amphiphilic nature of the complex. The conjugate further preserves and enhances the fat-binding capacity of chitosan while allowing ALA to retain its insulin-sensitizing properties. The synergy between these components can help to optimize modulation of adiposity switching with greater efficacy than either component alone. The covalent amide bond formed in this complex also enhances mucosal adherence and prolongs intestinal duration, supporting a more sustained release of bioactive, insulin-sensitizing signals during digestion.

As noted, the active ingredients in the primary fat oxidation embodiment are combined in an enteric coated, alginic acid capsule. This coating serves as a barrier, ensuring that the active ingredients remain intact until they reach the small intestine, where it undergoes hydration, and forms a viscous gel matrix. This gel structure interacts with bile salts through ionic and hydrogen bonding, partially sequestering them within the gel network, slowing the diffusion of active ingredients, allowing for a sustained and localized release in the small intestine. This reduces fat emulsifying activity from the bile salt formation of micelles and enhances hypothalamic signaling modulation. Thus, with fewer micelles available for lipid solubilization, dietary fats remain largely unabsorbed and are excreted, which complements the fat-binding properties of the active ingredients. By preventing premature degradation, the coating thus improves the bioavailability of the active ingredients. The mucilaginous properties of alginic acid can further modulate the GI environment, contributing to optimal conditions for the synergies of fat binding, insulin sensitization, and metabolic rate enhancement for optimal hypothalamus signaling.

Alternatively, while alginic acid offers an exemplary embodiment of enteric protection, alternative coating systems (including but not limited to wax-based microencapsulation, liposomal carriers, layered tablets, or the inclusion of recent specialized coating strategies, such as DehydraTECH™ (US 2021/0177978 A1), can be used to optimize ingredient stability, extend release profiles and enhance overall efficacy of the hypothalamic adiposity-switching mechanism.

Additional elements, included but not limited to anti-absorption agents and emulsification inhibitors, may be optionally integrated into other embodiments to reduce dietary fat absorption further, creating mechanisms that enhance fat oxidation and minimize fat bioavailability. This may be achieved by incorporating one or more non-digestible lipid-binding polymers, such as high-molecular-weight, non-absorbable polysaccharides, or by including a lipase inhibitor. Such formulations that further comprise an anti-absorption agent would bind dietary lipids by creating macromolecular aggregates that are too large for intestinal absorption. Likewise, by inhibiting their emulsification or enzymatic hydrolysis, such agents would reduce fat absorption, promote its undigested excretion and further reduce the net caloric uptake from fat.

In the preferred embodiment of the present invention for adiposity accumulation, the supplement comprises:

| Microencapsulated octanoic (C8) and | 4-10 | g/day | Provides the substrate for ghrelin acylation, elevates |
|---|---|---|---|
| decanoic (C10) MCTs | | | orexigenic acyl-ghrelin and appetite. |
| Structured triacyl-glycerols enriched | 2-6 | g/day | Maximizes intestinal fat absorption, supports positive |
| in sn-2 palmitate | | | nitrogen balance, and promotes weight gain. |
| Palmitoleic-acid-fortified olive oil | 1-4 | g/day | Acts as an insulin-sensitizing lipokine that channels |
| | | | nutrients into adipose tissue while limiting ectopic lipid |
| | | | deposition. |
| Essential phospholipids | 1-2 | g/day | Supplies phosphatidylcholine and related phospholipids |
| | | | that emulsify dietary fats, stabilizes cell membranes and |
| | | | aids lipid transport. |
| High-purity EPA/DHA | 3-4 | g/day | Blunts inflammatory catabolism, preserves muscle, and |

mitigates re-feeding-syndrome-like lipid derangements.

This embodiment spans a dosing range that can be adjusted for age, clinical status, or target rate of weight gain. The utility is to signal the hypothalamus to stimulate the energy-balance set-point toward storage and anabolism through a synergistic mechanism that is not taught by the prior art. Dose ranges can be titrated by body weight (mg/kg), goal, and divided into AM/PM administrations.

The mechanism of action for fat accumulation comprises four coordinated elements that span the small intestines to optimize hypothalamus signaling: 1) Ghrelin Amplification: Octanoic (C8) and decanoic (C10) MCTs are substrates for ghrelin-O-acyl transferase. Rapid duodenal MCT release elevates circulating acyl-ghrelin, may stimulate appetite, suppresses thermogenesis, and shifts hypothalamic neuropeptide expression toward NPY/AgRP, thereby initiating anabolic metabolism. 2) Efficient Energy Deposition: Structured triglycerides enriched in sn-2 palmitate mimic human-milk triglyceride architecture and maximize intestinal fat absorption to promote positive nitrogen levels. Co-delivery to the mid-jejunum with palmitoleic-acid fortified olive oil functions as an insulin-sensitizing lipokine that steers nutrients into adipocytes while limiting ectopic lipid liver deposition. 3) Lipid Assimilation and Transport: Essential phospholipids (predominantly phosphatidylcholine) also delivered to the mid-jejunum emulsify dietary fats in situ, stabilize chylomicron formation and support membrane biosynthesis during rapid tissue expansion. 4) Anti-catabolic Defense: High-purity EPA/DHA delivered to the distal ileum blunt inflammatory signaling, reduce proteolysis and prevent hepatic lipid overload and minimize the metabolic instability that can accompany aggressive re-feeding.

Unlike the fat-oxidation formulation, which leverages alginate coatings and optional lipid-scavenging polymers to limit fat uptake, this adiposity-accumulation embodiment preserves and enhances dietary lipid emulsification and absorption. No bile-sequestering agents, fat-binding polymers or lipase inhibitors are included. Each structural feature (phospholipid co-crystals, self-emulsifying excipients, pH-timed release) would be optimized for intestinal assimilation and channeling of nutrients into anabolic pathways.

To achieve targeted delivery, the capsule is engineered as a tri-layer, pH-responsive system that releases its active ingredients sequentially along and throughout the small intestines where hypothalamus signaling occurs. First, an outer duodenal primer layer coated with Eudragit L (dissolution threshold≥pH 5.5) would release microencapsulated C8/C10 MCT in the duodenum; these micron-sized beads are formulated with a self-emulsifying lipid excipient that accelerates absorption and triggers ghrelin acylation within minutes of ingestion. A stable oil-in-water emulsion is formed using MCT oil as the core material and gelatin as the primary wall material, optionally in the presence of Tween® 80 or similar emulsifiers. This emulsion is then subjected to a high-energy homogenization process to achieve uniform droplet size at relatively low surfactant-to-oil ratios. The resulting emulsion is spray-dried, producing microencapsulated MCT suitable for the duodenal primer layer. Such methods, conventionally practiced and well known to those skilled in the art of microencapsulation, enable consistent encapsulation of bioactive lipids, optimize stability and facilitate rapid re-emulsification in the intestinal environment. Second, a jejunal release layer would be protected by a Eudragit L100-55/S blend (≥pH 6.0) to deliver structured triacylglycerols enriched in sn-2 palmitate together with palmitoleic-acid-fortified olive oil and essential phospholipids. These lipids would be co-spray-dried into a lipid-phospholipid co-crystal that resists gastric lipase yet dissolves rapidly in bile-rich jejunal fluid, maximizing fat uptake and insulin-sensitizing signaling. Third, a distal-ileum anti-catabolic release layer coated with Eudragit S (≥pH 7.0) releases EPA/DHA embedded in an antioxidant-stabilized wax matrix that prevents peroxidation and provides a sustained, ileal delivery mechanism. This 3-layer phased design recreates the natural rostro-caudal nutrient-sensing gradient of the small intestine, supplying discrete hormonal and metabolic cues in the precise temporal order required to shift the hypothalamic set-point toward energy storage and anabolism.

Further novelty is introduced by the optional integration of supplementary agents, including but not limited to micronutrients, bioactive peptides, or modulators of gut hormone release (e.g., peptide YY or ghrelin analogs), into the formulation. Such inclusions can further refine the gut-brain signaling axis, thereby enhancing the efficacy of the adiposity switching mechanism.

What is claimed:

1. A nutraceutical composition for manipulating hypothalamic adiposity switching in a subject, the composition comprising an orally administered capsule or tablet, wherein the capsule or tablet comprises a formulation selected from the group consisting of:
(a) a fat oxidation formulation comprising:
(i) conjugated linoleic acid;
(ii) a sequestration component comprising chitosan optionally complexed with alpha-lipoic acid;
(iii) a fiber component comprising psyllium husk or a functional analog; and
(iv) an enteric coating comprising alginic acid or a functional analog;
(b) a fat accumulation formulation comprising:
(i) microencapsulated octanoic (C8) and decanoic (C10) medium-chain triglycerides;
(ii) structured triacylglycerols enriched in sn2 palmitate;
(iii) palmitoleic acid-fortified olive oil;
(iv) essential phospholipids; and
(v) high purity EPA/DHA;
wherein the composition is configured to release the selected formulation in the small intestine, thereby stimulating the hypothalamus to toggle between fat accumulation or fat oxidation in response to the presence or absence of lipid stimuli.

2. The composition of claim 1, wherein the components function as a bioregulator to restore hypothalamic homeostasis in the presence of metabolic dysregulation caused by caloric excess or deficiency, pharmaceuticals, or environmental factors.

3. The composition of claim 1, wherein the lipid-based and sequestration components are formulated to support personalized weight gain or weight loss protocols in individuals experiencing dysregulation, including but not limited to cachexia, obesity, or peri-post-operative metabolic needs.

4. The composition of claim 1, wherein the fat oxidation formulation is adapted to accommodate culturally diverse eating practices and used as an adjunct to weight loss plans.

5. The composition of claim 1, further comprising an anti-absorption agent selected from the group consisting of non-digestible lipid-binding polymers or lipase inhibitors to reduce dietary fat absorption and synergistically promote fat oxidation.

6. The composition of claim 1, wherein the enteric-coated delivery system enables gastrointestinal signaling over a prolonged period of days or weeks, sufficient to induce hypothalamic switching and overcome metabolic resistance.

7. The composition of claim 1, wherein the enteric or protective coating is selected from the group including but not limited to alginic acid, wax-based microencapsulation, liposomal carriers, layered tablet systems or similar coating strategies.

8. The composition of claim 1, wherein the composition is further operable to ameliorate eating pattern dysrhythmias, including but not limited to chronic restriction, binge behavior, or loss of appetite due to psychological and/or physiological causes.

9. The composition of claim 1, wherein the targeted hypothalamic signaling mechanism is configured to provide therapeutic benefit in wasting disorders including but not limited to cachexia, chemotherapy-induced weight loss, and chronic undernourishment syndromes.

10. The composition of claim 1, wherein the fiber component functions to modulate gastrointestinal transit time and enhance nutrient-sensing by enteroendocrine cells, thereby reinforcing hypothalamic lipid signaling.

11. The composition of claim 1, wherein the chitosan-ALA complex is formed via covalent amide bond linkage, resulting in increased intestinal retention time and controlled release of insulin-sensitizing activity.

12. The composition of claim 1, wherein the alginic acid enteric coating forms a gel matrix upon exposure to bile salts, sequestering micelles and reducing emulsification of dietary fats.

13. The composition of claim 1, wherein the fat oxidation formulation comprises conjugated linoleic acid at a dosage range of 0.5 g to 3.0 g per day, combined with a sequestration component comprising chitosan complexed with alpha-lipoic acid at a dosage range of about 0.25 g to about 1.0 g per day and about 0.30 g to about 0.60 g per day, respectively, and a fiber component comprising psyllium husk or a functional analog at a dosage range of about 5.0 g to about 10.0 g per day, collectively being sufficient to stimulate a hypothalamic response.

14. The composition of claim 1, wherein the chitosan-ALA complex is formed by activating the carboxyl groups of alpha-lipoic acid via carbodiimide-mediated coupling with the primary amine groups of chitosan to form covalent amide bonds.

15. The composition of claim 1, wherein the fat accumulation formulation comprises microencapsulated octanoic (C8) and decanoic (C10) medium-chain triglycerides present in a dosage range of 4 g to 10 g per day, structured triacylglycerols enriched in sn2 palmitate present in a dosage range of 2 g to 6 g per day, palmitoleic acid-fortified olive oil present in a dosage range of 1 g to 4 g per day, essential phospholipids present in a dosage range of 1 g to 2 g per day, and high purity EPA/DHA present in a dosage range of 3 g to 4 g per day, collectively being sufficient to stimulate a hypothalamic response.

16. The composition of claim 1, wherein the microencapsulated octanoic (C8) and decanoic (C10) medium-chain triglycerides is formed by preparing a stable oil-in-water emulsion comprising octanoic (C8) and decanoic (C10) medium-chain triglycerides oil, gelatin and Tween® 80 or similar emulsifiers, followed by high-energy homogenization and subsequent spray-drying to yield micron-sized beads.

17. The composition of claim 1, wherein the components of the fat oxidation formulation are microencapsulated and arranged in a tri-layer, pH-responsive system configured to sequentially release active ingredients along the small intestine, including: a duodenal primer layer coated with Eudragit® L for initial release, a jejunal release layer protected by a Eudragit® L100 55/S blend, and a distal ileum release layer coated with Eudragit® S, thereby mimicking intermittent lipid-signaling rhythms and optimizing hypothalamic modulation of adiposity switching.

18. The composition of claim 1, further comprising at least one anti-absorption or emulsification-inhibiting agent selected from the group consisting of non-digestible lipid-binding polymers, high-molecular-weight polysaccharides and lipase inhibitors.

19. The composition of claim 1, further comprising one or more supplementary agents selected from the group consisting of micronutrients, bioactive peptides, and gut hormone modulators.

20. The composition of claim 1, wherein the dosage regimen is provided in a multi-dose format and the dose ranges are titrated based on body weight (mg/kg), permitting flexible administration schedules that are customized to specific metabolic requirements.
